(19) United States
(12) Patent Application Publication
(10) Pub. No.: US 2012/0213717 A1
(43) Pub. Date: Aug. 23, 2012

(54) Soothing Agents

(75) Inventor: Mandar V. Shah, Rockaway, NJ (US)

(73) Assignee: McNeil-PPC, Inc., Skillman, NJ (US)

(21) Appl. No.: 13/030,305

(22) Filed: Feb. 18, 2011

(51) Int. Cl. A61K 31/60; A61P 11/00; A61Q 17/04; A61P 27/00; A61P 15/02; A61P 43/00; A61K 8/58; A61P 17/00

(52) U.S. Cl. 424/59; 514/164

(57) ABSTRACT

In certain embodiments, the present invention is directed to the use of soothing agents to relieve mild irritation and/or enhance the overall soothing, comfort or refreshing feel of mucosal membranes of the eye, oral cavity, otic, nose, throat or vaginal area. The present invention further relates to compositions, articles and methods for masking or reducing the irritant properties of sensory compounds, such as terpenes and/or phenolic agents.

### TECHNICAL FIELD

The present invention is directed to the use of soothing agents to relieve mild irritation and/or enhance the overall soothing, comfort or refreshing feel of mucosal membranes of the eye, oral cavity, otic, nose, throat or vaginal area. The present invention further relates to compositions, articles and methods for masking or reducing the irritant properties of sensory compounds, including terpenes (such as menthol and camphor) and/or phenolic agents (such as thymol) and the like.

### BACKGROUND OF THE INVENTION

A wide range of products incorporate ingredients which impart some noticeable sensation to the mucous membranes of the eye, otic cavity, nasal cavity, oral cavity, throat or vaginal area. These ingredients have most typically taken the form of sensory compounds, flavors or fragrances in a wide range of products such as personal care products (perfumes deodorants, cosmetics, shampoos, skin creams, toothpastes and the like), pharmaceuticals (such as cough syrups, cough drops and the like) and foods (such as chewing gum, soda and the like).

One sensory compound which is particularly useful for this purpose is menthol. Menthol is known as a coolant or cooling agent because of the cooling sensation it imparts to the skin and mucosal surfaces. Though widely used for this purpose, menthol is occasionally perceived as being irritating and consequently, has not been utilized as extensively in preparations for sensitive mucosal surfaces such as the eye, nasal cavity, vaginal area, etc.

The present inventor has discovered compounds useful in providing an improved sensory sensation, that is, a soothing type of sensation free of or substantially free of irritancy. Additionally, the compounds of the present invention improves the initial sensation experienced by users of sensory compounds such as menthol by masking or moderating the harsh and/or unpleasant effects compounds, without affecting cooling or other sensory benefits.

It is, therefore, an aspect of the present invention to provide compositions and/or articles which relieve mild irritation and/or enhance the overall comfort of the skin and mucosal membranes of the eye, otic cavity, nasal cavity, oral cavity, throat or vaginal area by incorporating at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

Another aspect of the present invention is to provide topical compositions and/or articles comprising: i.) at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon and a second hydroxyl group is attached to a second carbon of the carbon backbone adjacent to the first carbon and ii.) at least one sensory compound.

A further aspect of the present invention is to provide methods of masking or reducing the irritant properties of sensory compounds by applying separate to, or in the same formulation with, the sensory compound at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

Yet another aspect of the present invention is provide non-irritating compositions in the form of ophthalmic drops, mouthwashes, otic drops, skin or hair tonics, shampoos and conditioners comprising at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

### SUMMARY OF THE INVENTION

In certain embodiments, the present invention relates to compositions comprising from 0.05% (or about 0.05%) to less than about 5%, or, optionally, from 0.1% (or about 0.1%) to 3% (or about 3%), or, optionally, from 0.2% (or about 0.2%) to 1% (or about 1%) by weight of the total composition of at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

In other embodiments, the present invention relates to topical compositions providing a soothing or calming type of sensation comprising at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

In another embodiment, the present invention relates to methods of reducing harsh or unpleasant sensations associated with the topical application of sensory compounds to an host's skin or mucosal membranes of the eye, otic cavity, nasal cavity, oral cavity, throat or vaginal area, comprising the step of:
a. providing a topical composition comprising:
   i. a topical carrier,
   ii. at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon; and
   iii. at least one sensory compound; and
b. applying the topical composition to the host's skin or mucosal membranes of the eye, oral cavity, nose, throat or vaginal area.

In another embodiment, the present invention relates to methods of reducing harsh or unpleasant sensations associated with the topical application of sensory compounds to a host's skin or mucosal membranes of the eye, otic cavity, nasal cavity, oral cavity, throat, vaginal area comprising the step of:
a. applying to the host's skin or mucosal membranes of the eye, oral cavity, nose, throat or vaginal area at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon; and
b. applying to the host's skin or mucosal membranes of the eye, oral cavity, nose, throat or vaginal area at least one sensory compound.

In other embodiments, the compositions of the present invention relate to methods of reducing harsh or unpleasant sensations associated with inadvertent application of topical skin care compositions to a host's mucosal membranes of the eye, otic cavity, nasal cavity, oral cavity, throat or vaginal area comprising the step of applying a topical skin care composition comprising at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group attached a second carbon of the carbon backbone adjacent to the first carbon.

In still other embodiments, the present invention relates to articles comprising i.) at least one soothing agent selected from the group consisting of saturated or unsaturated, straight or branched chain alkyls having a carbon backbone and having no more than 5 carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; and ii.) optionally, at least one sensory compound.

In still further embodiments, the present invention relates to methods of reducing harsh or unpleasant sensations associated with the topical application of sensory compounds to an host's skin or mucosal membranes of the eye, otic cavity, nasal cavity, oral cavity, throat or vaginal area, comprising the step of:
a. providing a article comprising:
   i. at least one soothing agent selected from the group consisting of saturated or unsaturated, straight or branched chain alkyls having a carbon backbone and having no more than 5 carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; and
   ii. at least one sensory compound; and
b. applying the article to the host's skin or mucosal membranes of the eye, oral cavity, nose, throat or vaginal area.

### DETAILED DESCRIPTION OF THE INVENTION

The compositions of the present invention can comprise, consist of, or consist essentially of the essential elements and limitations of the invention described herein, as well any of the additional or optional ingredients, components, or limitations described herein. The term “comprising” (and its grammatical variations) as used herein is used in the inclusive sense of “having” or “including” and not in the exclusive sense of “consisting only of.”

The term ‘host” as used herein means humans or animals.

The terms “a” and “the” as used herein are understood to encompass the plural as well as the singular.

The phrase “visually clear”, as used herein means translucent to the unaided eye such that: the composition is i.) free of or substantially free of haziness and/or ii.) free of or substantially free of undissolved particles when viewed by the unaided eye.

Unless otherwise indicated, all documents cited are incorporated herein by reference; the citation of any document is not to be construed as an admission that it is prior art with response to the present invention. Furthermore, all documents incorporated herein by reference in their entirety are only incorporated herein to the extent that they are not inconsistent with this specification.

All percentages, parts and ratios are based upon the total weight (w/w) of the composition of the present invention, unless otherwise specified. All such weights as they pertain to the listed ingredients are based on the level of the particular ingredient described and, therefore, do not include carriers or by-products that may be included in commercially available materials, unless otherwise specified.

As used herein, the phrase “room temperature” shall mean 25° C. or about 25° C.

As used herein the phrase “terminal carbon” means the end carbon of a chain of carbon atoms or the end carbon of a straight chain alkyl.

The phrase “dermatologically acceptable carrier” means that the carrier is suitable for topical application to the keratinous tissue, has good aesthetic properties, is compatible with the actives of the present invention and any other components, and will not cause any safety or toxicity concerns.

The phrase “nasally acceptable carrier” means that the carrier is suitable for topical application to the mucosal tissues or membranes of the inner nose, is compatible with the actives of the present invention and any other components, and will not cause any safety or toxicity concerns.

The phrase “otically acceptable carrier” means that the carrier is suitable for topical application to the mucosal tissues or membranes of the inner ear, is compatible with the actives of the present invention and any other components, and will not cause any safety or toxicity concerns.

The phrase “ophthalmologically acceptable carrier” means that the carrier is suitable for topical application to the mucosal tissues or membranes of the eye, is compatible with the actives of the present invention and any other components, and will not cause any safety or toxicity concerns.

The phrase “vaginally acceptable carrier” means that the carrier is suitable for topical application to the mucosal tissues or membranes of the peri-vaginal or inner vaginal area, is compatible with the actives of the present invention and any other components, and will not cause any safety or toxicity concerns.

The phrase “orally acceptable carrier” means that the carrier is suitable for application to the surfaces of the oral cavity by a living organism including, but not limited to, mammals and humans without undue toxicity, incompatibility, instability, allergic response, and the like.

### Soothing Agent

The compositions of the present invention comprise at least one soothing agent selected from the group consisting of saturated or unsaturated, straight or branched chain, aliphatic or non-aliphatic, alkyls having a carbon backbone of no more than 5 carbons in length, optionally, no more than 4 carbons in length, and wherein the alkyl has at least two hydroxyl groups such that a first hydroxyl group is attached to a first carbon of the carbon backbone and a second hydroxyl group is attached to a second carbon of the carbon backbone adjacent to the first carbon.

In certain embodiments, the compositions of the present invention comprise at least one soothing agent selected from the group consisting of saturated or unsaturated, straight chain, aliphatic alkyls having a carbon chain length of from 4 to 5 carbons and having at least two hydroxyl groups such that a first hydroxyl group is attached to a terminal carbon of the straight chain alkyl and a second hydroxyl group is attached to a carbon of the straight chain of the alkyl which is adjacent to the terminal carbon of the straight chain alkyl.

In certain embodiments, the soothing agent is selected from the group consisting of alkanediols, alkanetriols or mixtures thereof. In certain embodiments, the soothing agent is an alkanediol or mixtures thereof. In certain embodiments, the soothing agent is an alkanetriol or mixtures thereof. Suitable alkanediols include, but are not limited to, 1,2 butanediol; 1,2 pentanediol; 2,3 butanediol; 2,3 pentanediol; 3,4 pentanediol and mixtures thereof. In certain embodiments, the alkanediols are selected from the group consisting of 1, 2 butanediol; 1,2 pentanediol and mixtures thereof. Suitable alkanetriols include, but are not limited to, 1,2,3 butanetriol; 1,2,3 pentanetriol; 2,3,4 pentanetriol, 1,2,4 butanetriol; 1,2,4 pentanetriol and mixtures thereof, In certain embodiments, the alkanetriols are selected from the group consisting of 1,2,3 butanetriol; 1,2,3 pentanetriol and mixtures thereof. Mixtures the above diols and triols can also be used herein. In some embodiments, the soothing agent is selected from the group consisting of, 1,2 butanediol; 1,2 pentanediol; 1,2,3 butanetriol; 1,2,3 pentanetriol; and mixtures thereof. In other embodiments, the soothing agent is selected from the group consisting of, 1,2 pentanediol; 1,2,3 butanetriol; 1,2,3 pentanetriol; 1,2,4 butanetriol; 1,2,4 pentanetriol and mixtures thereof. In still other embodiments, chemical derivatives of any of the above mentioned compounds may also be used so long as such chemical derivatization does not affect the functional properties of adjacent hydroxyl groups of the compounds. Without being limited by theory, it is believed that the functional properties of the adjacent of the hydroxyl groups aid in providing the soothing effects of the soothing agents.

Soothing Agents suitable for use herein can be obtained from Sigma-Aldrich, St. Louis, Mo.

In certain skin care embodiments of the present invention, the soothing agent is incorporated at concentrations of from 0.05% (or about 0.05%) to less than about 5%, or, optionally, from 0.1% (or about 0.1%) to 3% (or about 3%), or, optionally, from 0.2% (or about 0.2%) to 1% (or about 1%) by weight of the total composition.

In certain eye care, oral care, nasal care, otic care, and/or vaginal care embodiments of the present invention, the soothing agent is incorporated at concentrations of from 0.05% (or about 0.05%) to 10% (or about 10%), or, optionally, from 0.1% (or about 0.1%) to 5% (or about or less than about 5%), or, or, optionally, from 0.2% (or about 0.2%) to 3% (or about 3%), or, optionally, from 0.2% (or about 0.2%) to 1% (or about 1%) by weight of the total composition.

The soothing agents of the present invention may also be incorporated into compositions which are not intended for use in the eye, otic cavity, nasal cavity, oral cavity, throat, vaginal area, but may inadvertently be instilled in such areas. Such compositions include bodywashes, soaps, skin or hair lotions, skin or hair sprays, sunscreens, shampoos and conditioners which may inadvertently be instilled in sensitive mucosal areas (such as the eyes or vaginal area), causing irritation.

### Optional Ingredients

### Sensory Compound

In certain aspects the compositions of the present invention may optionally comprise a sensory compound. Sensory compounds of the present invention include, but are not limited to, terpenes, phenolic compounds and mixtures thereof.

In certain embodiments, the sensory compounds include, but are not limited to, terpenes such as neral (citral B), nerol, citral, phytol,alpha-pinene, beta-pinene, camphor, limonene, menthol, geranial (citral A), geraniol, farnesol and mixtures thereof A more detailed discussion of terpenes can be found in U.S. Pat. No. 7,727,516 B2 to Botchikareva et al., herein incorporated by reference in its entirety.

In other embodiments, the sensory compounds include, but are not limited to, phenolic compounds such as thymol, methyl salicylate, 1,8-cineol (eucalyptol) and mixtures thereof. A more detailed discussion of useful phenolic compounds can be found in U.S. Pat. No. 5,344,641 to Gaffar et al., herein incorporated by reference in its entirety.

In certain embodiments, the compositions of the present invention comprise a sensory compound selected from the group consisting of menthol, camphor, borneol, geraniol and mixtures thereof. In certain other embodiments, the compositions of the present invention comprise a sensory compound selected from the group consisting of menthol, thymol, methyl salicylate, 1,8-cineol (eucalyptol) and mixtures thereof.

Sensory compounds suitable for use herein can be obtained from Sigma-Aldrich, St. Louis, Mo., St. Louis, Mo. Useful sensory compounds can also be obtained from Symrise Inc., Somerville, N.J.; or from Jindal Drugs Limited, Mumbai, India; or from Rhodia Inc., Cranbury, N.J.

In certain skin care embodiments, when incorporated in the same composition with (or applied sequentially with) the soothing agent of the present invention, the soothing agent and the sensory compound are present in the composition (or applied) at a ratio of soothing agent to sensory compound of from 1:2 (or about 1:2) to 1:20 (or about 1:20), or optionally from 1:7 (or about 1:7) to 1:15 (or about 1:15) by weight. These embodiments optionally comprise a dermatologically acceptable carrier.

In certain oral care embodiments, when incorporated in the same composition with (or applied sequentially with) the soothing agent of the present invention, the soothing agent and the sensory compound are present in the composition (or applied) at a ratio of soothing agent to sensory compound of from 5:1 (or about 5:1) to 1:5 (or about 1:5), or optionally from 3:1 (or about 3:1) to 1:3 (or about 1:3), or optionally from 3:1 (or about 3:1) to 1:1 (or about 1:1) by weight. These embodiments optionally comprise an orally acceptable carrier.

In certain eye care, nasal care and otic care embodiments, when incorporated in the same composition with (or applied sequentially with) the soothing agent of the present invention, the soothing agent and the sensory compound are present in the composition (or applied) at a ratio of soothing agent to sensory compound of from 175:1 (or about 175:1) to 25:1 (or about 25:1), or optionally 150:1 (or about 150:1) to 50:1 (or about 50:1), or optionally from 125:1 (or about 125:1) to 75:1 (or about 75:1) by weight. These embodiments optionally comprise an ophthalmologically acceptable, a nasally acceptable carrier, or an otically acceptable carrier, respectively.

In certain vaginal care embodiments, when incorporated in the same composition with (or applied sequentially with) the soothing agent of the present invention, the soothing agent and the sensory compound are present in the composition (or applied) at a ratio of soothing agent to sensory compound of from 17.5:1 (or about 17.5:1) to 2.5:1 (or about 2.5:1), or optionally 15.0:1 (or about 150:1) to 5:1 (or about 5:1), or optionally from 12.5:1 (or about 12.5:1) to 7.5:1 (or about 7.5:1) by weight. These embodiments optionally comprise a vaginally acceptable carrier.

### Carriers and Carrier Components

In certain embodiments, the compositions of the present invention optionally include a topical carrier or vehicle for delivering the soothing agent and any optional ingredients. When used herein, the term “topical” includes reference to formulations that are suitable for application to a host's outer body surfaces (e.g. the skin or mucous surfaces of the body, including oral, eye, nasal, otic or vaginal surfaces), but not for (or intended for) ingestion for systemic purposes (such as for satiety or any other caloric purposes and/or pharmacologic purposes). In some embodiments, the soothing and/or irritation reducing/preventing effects caused by the topical application of the soothing agents of the present invention are localized to the area of application.

Mucous membranes that may be mentioned in this respect include the mucosa of the vagina, the penis, the urethra, the anus, the mouth (including the mucosa of the cheek, the soft palate, the under surface of tongue and the floor of the mouth), the nose, the throat (including the mucosa of the pharynx, the larynx, the trachea and the esophagus), the eye and the ear.

Thus, in certain embodiments of the present invention, the topical composition include, but is not limited to, topical pain relieving or analgesic, (such as topical analgesic ointments or creams), oral care (such as a mouthrinse, or oral dentifrice), intranasal, intra- or peri-vaginal (such as creams, suppositories, lotions or sprays), ophthalmic compositions (such as eyedrops and eye ointments), topical bases (such as, but not limited to, shampoo, bodywash, soap, lotion foam, solution, or sunscreen bases) or articles (such as topically applied fabrics, adhesive patches or coverings).

Topical compositions, which are useful for treating disorders of the skin or of mucous or mucosal membranes (e.g. those accessible by digitation, such as membranes of the mouth, vagina, cervix, anus and rectum), include creams, ointments, lotions, liniments, sprays, gels and sterile aqueous solutions or suspensions. As such, topical compositions include those in which the active ingredient(s) is (are) dissolved or dispersed in a dermatologically, nasally, otically, ophthalmologically, vaginally or orally acceptable carrier as known in the art (e.g. alcoholic, aqueous, hydroalcoholic, or non-aqueous solutions; aqueous or non-aqueous gels; ointments; or water-in-oil or oil-in-water emulsions). Constituents of such carriers or vehicles may comprise water, aqueous buffer solutions, non-aqueous solvents (such as ethanol, isopropanol, benzyl alcohol, 2-(2-ethoxyethoxy) ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone) and mixtures of any of the above mentioned ingredients. Depending, inter alia, upon the nature of the formulation as well as its intended use and site of application, the topical carrier or vehicle employed may contain one or more components (for example, when the formulation is an aqueous gel, components in addition to water) selected from the following list: a solubilising agent or solvent (e.g. a (3-cyclodextrin, such as hydroxypropyl P-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

The carrier of the present invention can also be in the form of an article for application to the skin or mucous membranes (including insertion into skin and/or mucosal cavities) of a host. In certain embodiments, the soothing agents and/or sensory compounds can be layered onto or infused into such articles. Suitable examples of such articles include, but are not limited to, cleaning wipes, tissues or pads (including cosmetic wipes, tissues or pads and wipes; tissues or pads used for cleaning the area around the eye and baby wipes, tissues and pads), disposable absorbent articles, adhesive articles, wraps and protectant or light shielding covers and the like. Examples of such cleansing wipe, tissue or pad articles include, but are not limited to, wet or dry packaged, nonwoven articles. Examples of such disposable absorbent articles include, but are not limited to, health care related products including bandages and tampons such as those intended for medical, dental, surgical and/or nasal use; personal care absorbent products such as feminine hygiene products (e.g., sanitary napkins, panty liners, and catamenial tampons), diapers, training pants, incontinent products and the like. Examples of adhesive articles include, but are not limited to, patches or articles which are singles layer or multilayer sheets comprising (or at least one of which layers comprises) woven and/or nonwoven materials where the adhesive articles can be semi-occlusive or non-occlusive. Examples of such wraps articles include, but are not limited to, elastic or non-elastic, woven or nonwoven fabric materials. Examples of such analgesic or light shielding eye covers include, but are not limited to, eye mask/covers for: i) sleeping; ii) retaining the eyelid in a closed position; or iii) relieving achy, irritated eyes or headaches.

A more detailed discussion of useful articles, carriers or vehicles and/or carrier or vehicle ingredients can be found in U.S. Pat. No. 7,118,759 B2 to Syverson et al.; U.S. Pat. No.5,536,263 to Rolf et al.; U.S. Pat. No. 5,860,945 to Cramer et al.; U.S. Pat. No. 6,440,437 to Krzysik et al.; U.S. Pat. No. 6,121,315 A to Nair et al.; U.S. Pat. No. 7,235,249 B2 to Bissett; U.S. Pat. No. 7,671,206 B2 to Gómez et al. and U.S. Pat.Publ. 20100226963 Al to Cooper et al., each of which documents is herein incorporated by reference in its entirety.

In certain embodiments, the compositions and/or articles of the present invention are free of, or essentially free of compounds which enhance skin penetration of active agents or any other ingredients which might elicit unintended and/or adverse systemic effects on a host. As used herein, “skin penetration enhancing compounds”, means any compound or mixture of compounds that enhance skin penetration active agents when combined with the soothing agents of the present invention. “Essentially free” as used with respect to skin penetration enhancing compounds is defined as formulations having less than 10% (or about 10%), or optionally,5% (or about 5%), or optionally, 3% (or about 3%), or optionally, 1% (or about 1%), or optionally, 0.01% (or about 0.01%), or optionally, 0.001% (or about 0.001%), or optionally, 0.0001% (or about 0.0001%) by weight (w/v) of the total composition or (w/w) of the article of such skin penetration enhancing compounds. In certain embodiments, the skin penetration enhancing compounds include, but are not limited to, cell envelope-disordering compounds such as methyl myristate, methyl laurate, ethyl laurate, ethyl myristate, myristyl acetate, lauryl acetate and mixtures thereof; unsaturated fatty acid lipids such as oleic acid, myristoleic acid, palmitoleic acid, vaccenic acid, heptadecanoic acid, petroselenic acid, eicosenoic, linoelaidic acid linoleic acid and mixtures thereof; and mixtures thereof. In other embodiments, the compositions and/or articles of the present invention are free of or essentially free of cell envelope-disordering compounds such as methyl myristate, methyl laurate, ethyl laurate, ethyl myristate, myristyl acetate, lauryl acetate and mixtures thereof.

### Active Agents

Suitable active agents include drugs, prodrugs and nondrugs such as, but are not limited to, the pharmaceutical categories of anti-inflammatory agents, antiseptics, anti-infectives, mucous membrane agents, cleansing agents, preservatives, astringents, antihistamines, analgesics, capsaicin, acne medications, antifingals, antipruritics, hormones, growth factors, moisturizers, sunscreens, hyperpigmentation agents, antioxidants, nutritional substances, including, but not limited to, vitamins and minerals, substances of botanical, marine and animal origin, homeopathic substances and mixtures thereof.

A more detailed discussion of useful active agents can be found in previously incorporated by reference documents U.S. Pat. No. 6,121,315 A to Nair et al.; U.S. Pat. No. 7,235,249 B2 to Bissett; U.S. Pat. No. 7,671,206 B2 to GOrnez et al. and US Pat.Publ. 20100226963 Al to Cooper et al.

### EXAMPLES

The compositions of the present invention as described in following examples illustrate specific embodiments of compositions of the present invention, but are not intended to be limiting thereof. Other modifications can be undertaken by the skilled artisan without departing from the spirit and scope of this invention.

### Example I

Example I is an example of a topical arthritis greaseless cream containing 2,3 pentanediol as the soothing agent of the present invention and a sensory compound comprising menthol and methyl salicylate.

Topical Arthritis Greaseless Cream

| Ingredient | Amount (% w/w) |
|---|---|
| Methyl Salicylate¹ | 15.0 |
| Menthol, Racemic² | 10.0 |
| Steric Acid | 13.0 |
| Glyceryl Monosterate | 8.0 |
| Wool's Fat (lanolin anhydrous) | 2.0 |
| Sorbitan Trioleate (Tween 85) | 2.0 |
| Sorbitan tristearate (Span 65) | 1.0 |
| Triethanolamine | Adjust pH |
| 2,3 pentanediol³ | 5.0 |
| Purified Water | 43.0 |

¹Supplied by Rhodia Inc., Cranbury, NJ
²Supplied by Jindal Drugs Limited, Mumbai, India
³Supplied by Sigma-Aldrich, St. Louis, MO.

In a suitable vessel a lipid phase is prepared by adding the menthol and methyl salicylate while heating the vessel to a temperature of about 55° C. Once the menthol and methyl salicylate are melted, stearic acid, glyceryl monostearate, lanolin and sorbitan trioleate and sorbitan tristearate are added and the contents of the vessel is heated to about 73° C. and mixed well.

In a second vessel, an aqueous phase is prepared by adding water and heating the contents of to about 73° C. In the heated second vessel, 2,3 butanediol is added with mixing until it is dissolved.

The lipid phase and aqueous phases are mixed while maintaining a temperature of about 73° C. with mixing for about 20 minutes. The biphasic mixture is cooled to about 25° C. The pH of the mixture is adjusted to about 7.0 with triethanolamine, if needed, and any additional water is added as needed for volume. The mixture is then packaged in a suitable container.

### Example II

Example II is an example of a topical arthritis lotion containing 1,2 pentanediol as the soothing agent of the present invention and a sensory compound comprising menthol and methyl salicylate.

Topical Arthritis Lotion

| Ingredient | Amount (% w/w) |
|---|---|
| Steric Acid | 11.40 |
| Glyceryl Monosterate | 6.40 |
| Lanolin | 1.60 |
| Potassium Cetyl Phosphate | 0.22 |
| Menthol¹ | 8.00 |
| Methyl Salicylate² | 30.00 |
| 1,2 Pentanediol³ | 5.0 |
| Purified Water | 36.88 |
| Methylparaben | 0.25 |
| Propylparaben | 0.10 |
| Potassium Hydroxide | Adjust pH |

¹Supplied by Jindal Drugs Limited, Mumbai, India
²Supplied by Rhodia, Inc. Cranbury, NJ
³Supplied by ^{1.} Sigma-Aldrich, St. Louis, MO.

In a suitable vessel a lipid phase is prepared by adding the menthol and methyl salicylate while heating the vessel to a temperature of about 55° C. Once the menthol and methyl salicylate are melted, the stearic acid, glyceryl monostearate, lanolin and potassium cetyl phosphate are added and the contents of the vessel is heated to about 73° C. and mixed well.

In a second vessel, an aqueous phase is prepared by adding the water and heating the contents of the vessel to about 73° C. To the second vessel, 1,2 pentanediol, methylparaben and propylparaben are added with mixing until the ingredients are dissolved. The lipid phase and aqueous phases are mixed while maintaining a temperature of about 73° C. with mixing for about 20 minutes. The biphasic mixture is cooled to about 25° C. The pH of the mixture is adjusted to about 7.0 with potassium hydroxide, if needed, and any additional water is added as needed for volume. The mixture is then packaged in a suitable container.

### Example III

Example III is an example of a mouthwash containing 2,3 butanediol as the soothing agent of the present invention and a sensory compound comprising menthol, methyl salicylate, eucalyptol, and thymol.

Mouthwash

| Ingredient | Amount (% w/w) |
|---|---|
| Alcohol USP/EP | 22.6530 |
| Menthol USP¹ | 0.323 |
| Thymol NF² | 0.0639 |
| Methyl Salicylate NF³ | 0.0660 |
| Eucalyptol USP⁴ | 0.0922 |
| Mint Flavor | 0.1 |
| 2,3 Butanediol⁵ | 1.0% |
| Poloxamer 407 | 0.2500 |
| Sorbitol Solution, 70% | 20.00 |
| Benzoic Acid USP | 0.1200 |
| Sodium Benzoate NF | 0.0354 |
| Saccharin Sodium USP | 0.12 |
| Appropriate color | 0.0005 |
| Purified Water | Q.S |

¹Supplied by Jindal Drugs Limited, Mumbai, India
²Supplied by Symrise Inc., Somerville, NJ
³Supplied by Rhodia Inc., Cranbury, NJ
⁴Supplied by Jindal Drugs Limited, Mumbai, India
⁵Supplied by Sigma-Aldrich, St. Louis, MO.

In a suitable vessel, a mixture is prepared by adding the alcohol, menthol, thymol, methyl salicylate and eucalyptol, flavor and mixed until homogeneous.

In another vessel, a second mixture is prepared by adding the water, 2,3 butanediol, benzoic acid and mixing the aqueous phase until they are dissolved. Then poloxamer, sorbitol, benzoate, saccharin and color are added into the second mixture which is aqueous and mixed until homogenous.

Then first mixture is combined with the second mixture of the second vessel to form a third mixture. The third mixture is mixed until homogenous and water is added as needed to adjust the volume. Once mixed, the third mixture is packaged in a suitable container.

### Example IV

Example IV is an example of an ophthalmic preparation containing 1,2 butanediol as the soothing agent of the present invention.

Ophthalmic Preparation

| Ingredient | Amount (% w/w) |
|---|---|
| Hydroxypropylmethylcellulose (HPMC) | 0.4 |
| 1,2 Butanediol¹ | 0.5 |
| Boric acid/sodium borate | 0.6 |
| Sodium citrate | 0.001 |
| Glycerin | 0.25 |
| Polyethylene glycol 400 (PEG) | 1.13 |
| Sodium Phosphate Dibasic | 0.03 |
| Potassium Chloride | 0.2 |
| Magnesium chloride | 0.013 |
| Sodium chloride | 0.07 |
| Dextrose | 0.004 |
| Sodium lactate | 0.05 |
| Glycine | 0.0002 |
| 1N HCl/NaOH | Adjust pH |
| Purified Water | QS to 100 |

¹Supplied by Sigma-Aldrich, St. Louis, MO.

In a suitable vessel, about 80 ml of water is added and heated to about 75° C. To the heated water, HPMC is added and dispersed well. Once the dispersion is homogenous, it is allowed to cool at broom temperature to about room temperature under stirring. The glycerin and PEG are added and mixed until homogeneous. The remaining ingredients are added and the mixture is mixed until visually clear, forming a solution. Once visually clear, the pH is adjusted to about 7.0 with 1N solution of HCI or NaOH, as needed, and any additional water is added as needed for volume. The solution is then packaged in a suitable container.

### Example V

Example V is an example of an ophthalmic preparation containing the compositions of the present invention, containing 1,2,4 butanetriol as the soothing agent and menthol as the sensory compound.

Ophthalmic Preparation

| Ingredient | Amount (% w/w) |
|---|---|
| Hydroxypropylmethylcellulose (HPMC) | 0.4 |
| 1,2,4 Butanetriol¹ | 0.5 |
| Boric acid/sodium borate | 0.6 |
| Sodium citrate | 0.001 |
| Glycerin | 0.25 |
| Polyethylene glycol 400 (PEG) | 1.13 |
| Menthol² | 0.005 |
| Sodium Phosphate Dibasic | 0.03 |
| Potassium Chloride | 0.2 |
| Magnesium chloride | 0.013 |
| Sodium chloride | 0.07 |
| Dextrose | 0.004 |
| Sodium lactate | 0.05 |
| Glycine | 0.0002 |
| 1N HCl/NaOH | Adjust pH |
| Purified Water | QS to 100 |

¹Supplied by Sigma-Aldrich, St. Louis, MO
²Supplied by Jindal Drugs Limited, Mumbai, India

In a suitable first vessel, about 80 ml of water is added and heated to about 75° C. To the heated lOwater, HPMC is added and mixed until homogeneous. Once the dispersion is homogenous, it is allowed cool at room temperature to about room temperature with stirring. Once cooled, the glycerin is added and mixed until homogeneous. In a suitable second vessel, the PEG is added. The menthol is added to the PEG and mixed until dissolved. The mixture of second vessel is added to mixture of the first vessel. The remaining ingredients are added to the first vessel and the mixture is mixed until visually clear, forming a solution. Once visually clear, the pH is adjusted to about 7.0 with 1N solution of HCI or NaOH, as needed, and any additional water is added as needed for volume. The solution is then packaged in a suitable container.

### Example VI

Example VI is an example of a contact lens cleaning solution, containing 2,3, butanediol as the soothing agent.

Contact Lens Cleaning Solution

| Ingredient | Amount (% w/w) |
|---|---|
| Polyvinyl pyrrolidone (PVP) | 1.5 |
| 2,3 Butanediol¹ | 0.5 |
| Boric acid/sodium borate | 0.6 |
| Sodium citrate | 0.1 |
| Poloxamer | 0.5 |
| Polyquaternium-42 | 0.005 |
| Ethylenediaminetetraaceticacid | 0.1 |
| (EDTA) | |
| Sodium chloride | 0.25 |
| 1N HCl/NaOH | Adjust pH |
| Purified Water | QS to 100 |

¹Supplied by Sigma-Aldrich, St. Louis, MO

In a suitable vessel, about 80 ml of water is added. The PVP is added to the water mixed until dissolved. The remaining ingredients are added, individually, with mixing such that each ingredient is dissolved before the adding the next ingredient, forming a solution. Once all the ingredients are added and the solution is mixed until visually clear, the pH is adjusted to about 7.0 with 1N solution 15of HCI or NaOH, as needed, and any additional water is added as needed for volume. The solution is then packaged in a suitable container.

### Example VII

Example VII is an example of a vaginal lubricant comprising 1,2 butanediol as the soothing agent and a the sensory compound comprising menthol and methyl salicylate.

Vaginal Lubricant

| Ingredient | Amount (% w/w) |
|---|---|
| Propylene glycol | 15.0 |
| Menthol¹ | 0.1 |
| Methyl Salicylate² | 0.1 |
| 1,2 Butanediol³ | 2.0 |
| Hydroxyethyl cellulose (HEC) | 0.75 |
| Benzoic Acid | 0.1 |
| Polyquaternium-1⁴ | 0.005 |
| Polysorbate 60 | 1.0 |
| Flavor/fragrance | 0.06 |
| 1N HCl/NaOH | Adjust pH |
| Purified Water | QS to 100 |

¹Supplied by Jindal Drugs Limited, Mumbai, India
²Supplied by Rhodia Inc., Cranbury, NJ
³Supplied by Sigma-Aldrich, St. Louis, MO.
⁴Supplied by Stepan Chemicals, Northfield IL, sold under the name, Onamer ® M

In a first suitable vessel, about 80 ml of the water is added and heated to about 75° C. To the heated water, HEC is added with mixing. Once the HEC mixture is homogenous, the mixture is cooled to room temperature while stirring the mixture. Once the mixture reaches room temperature, the benzoic acid and polyquaternium-1 is added and mixed until homogeneous. In a second suitable vessel, propylene glycol and polysorbate 60 are added and mixed until homogeneous. The menthol and methyl salicylate are added to the second vessel and mixed until dissolved. Once the mixture in the second vessel is visually clear, the mixture of the second vessel is added to the HEC mixture of the first vessel. The remaining ingredients are then added and the mixture is mixed until visually clear, forming a solution. The pH of the solution should be around 4.0, if not adjust it with 1N solution of HCI or NaOH, as needed. Any additional water is added as needed for volume adjustment. The solution is packaged in a suitable container.

### Example VIII

Example VIII is an example of a shampoo with conditioner, comprising 1,2 butanediol as the soothing agent, for reducing irritation to the eye upon inadvertant exposure.

Shampoo with Conditioner

| Ingredient | Amount (% w/w) |
|---|---|
| Polyquaternium 10 | 0.1 |
| Acrylamidopropyltrimonium | 0.03 |
| Chloride/Acrylamide copolymer | |
| (Salcare SC60) | |
| Polysorbate 20 | 5.0 |
| 1,2 Butanediol¹ | 1.0 |
| Sodium Laureth 2EO Sulfate 70% | 4.05 |
| Sodium Lauroamphoacetate | 5.9 |
| Cocamidopropyl Betaine | 1.0 |
| PPG-2-Hydroxyethyl cocamide | 0.5 |
| Cetyl triethylmonium dimethicone PEG-8 | 0.5 |
| PEG-150 Distearate | 1.7 |
| Fragrance | 0.2 |
| Polyquaternium 15 | 0.05 |
| Benzyl Alcohol | 0.1 |
| Color | 0.003 |
| Ethylenediaminetetraaceticacid (EDTA) | 0.08 |
| Citric acid | 0.35 |
| Purified Water | QS to 100 |

¹Supplied by Sigma-Aldrich, St. Louis, MO.

In a first vessel, about 50 ml of water is added and heated it to from about 80° C. to about 85° C. To the water, sodium lauroamphoacetate, EDTA and PEG-150 Distearate are added with mixing. The mixture is then mixed until uniform and homogeneous. Once the mixture is uniform and homogeneous, the polysorbate 20 added. The mixture is then mixed, dissolving the ingredients, until the mixture is visually clear, forming a solution. Sodium Laureth 2EO sulfate (70%) is then added to the solution and mixed for about 30 minutes. The solution is allowed to cool at room temperature. While the solution is cooling, the cocamidopropyl betaine, PPG-2-hydroxyethyl cocamide and cetyl triethylmonium dimethicone PEG-8, are added with constant mixing.

In a second vessel, a premix is prepared by adding Salcare SC60 and polyquaternium 10 to about 20 ml of water. The premix is mixed until visually clear, forming a solution. The premix solution of the second vessel is then added to the solution of the first vessel with mixing. The second vessel is rinsed with about 5 ml of water to capture any remaining premix. The premix rinse is then added to the first vessel. In the second vessel, the citric acid is added to about 5 ml of water with stirring to form a citric acid solution. The citric acid solution and then added it to the first vessel.

In a third vessel, the fragrance and the Tween 20 are mixed and then added to the first vessel. The polyquaternium 15 and benzyl alcohol are then, separately. added to the first vessel with mixing. The mixture of the first vessel is then mixed until all the added ingredients are dissolved, forming a solution.

In a forth vessel, the color is added to about 5 ml of water and then added to the solution of the first vessel. Next, the 1,2 butanediol is added to the solution of the first vessel and mixed until the solution is visually clear.

The pH of the solution is then adjusted to about 7.0 with additional citric acid, as needed, and any additional water is added as needed for volume. The solution is packaged in a suitable container.

1. A composition comprising from about 0.05% to less than about 5% of at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon and (ii) optionally, an alcoholic, aqueous, or hydroalcoholic topical carrier, wherein the composition is essentially free of compounds which enhance skin penetration of active agents.

2. The composition of claim 1 wherein the soothing agent is selected from the group consisting of alkanediols, alkanetriols and mixtures thereof

3. The composition of claim 2 wherein the alkanediol is selected from the group consisting of 1,2 butanediol; 2,3 butanediol; and mixtures thereof.

4. The composition of claim 3 wherein the alkanediol is 1,2 butanediol.

5. The composition of claim 2 wherein the alkanetriol is selected from the group consisting of, 1,2,3 butanetriol; 1,2,4 butanetriol, and mixtures thereof.

6. The composition of claim 5 wherein the alkanetriol is 1,2,3 butanetriol.

7. A topical composition for providing a soothing or a calming sensation comprising(i) at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; optionally, an alcoholic, aqueous, or hydroalcoholic topical carrier; and (iii) at least one sensory compound, wherein the composition is essentially free of compounds which enhance skin penetration of active agents.

8. The topical composition of claim 7, wherein the sensory compound is selected from the group consisting of terpenes, phenolic compounds and mixtures thereof.

9. The topical composition of claim 8, wherein the terpene is selected from the group consisting of neral, nerol, citral, phytol,alpha-pinene, beta-pinene, camphor, limonene, menthol, geranial, geraniol, farnesol and mixtures thereof

10. The topical composition of claim 8, wherein the phenolic compound is selected from the group consisting of thymol, methyl salicylate, eucalyptol and mixtures.

11. The topical composition of claim 8, wherein the sensory compound is selected from the group consisting menthol, camphor, borneol, geraniol and mixtures thereof.

12. The topical composition of claim 8, wherein the sensory compound is selected from the group consisting menthol, camphor, borneol, geraniol and mixtures thereof menthol, thymol, methyl salicylate, eucalyptol and mixtures thereof.

13. The topical composition of claim 7, wherein the topical composition is selected from the group consisting of skin compositions, analgesic compositions, ophthalmic compositions, otic compositions, nasal compositions and vaginal compositions.

14. A method of reducing harsh or unpleasant sensations associated with the topical application of sensory compounds to an host's skin or mucosal membranes of the eye, otic cavity, nasal cavity, oral cavity, throat or vaginal area, comprising the step of:
a. applying at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon, optionally, in an alcoholic, aqueous, or hydroalcoholic topical carrier; and
b. applying at least one sensory compound, optionally, in an alcoholic, aqueous, or hydroalcoholic topical carrier, wherein the host's skin or mucosal membranes are essentially free of compounds which enhance skin penetration of active agents.

15. A method of reducing harsh or unpleasant sensations associated with the topical application of sensory compounds to a host's skin or mucosal membranes of the eye, otic cavity, nasal cavity, oral cavity, throat or vaginal area , comprising the step of:
a. providing a topical composition comprising:
i. optionally, an alcoholic, aqueous, or hydroalcoholic topical carrier,
ii. at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; and
iii. at least one sensory compound
wherein the composition is essentially free of compounds which enhance skin penetration of active agents; and
b. applying the topical composition to the host's skin or mucosal membranes of the eye, oral cavity, nose, throat or vaginal area.

16. A method of reducing harsh or unpleasant sensations associated with inadvertent application of topical skin care compositions to a host's mucosal membranes of the eye, otic cavity, nasal cavity, oral cavity, throat or vaginal area comprising the step of applying a topical skin care composition comprising i.) at least one soothing agent selected from the group consisting of saturated or unsaturated, straight -or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; and ii.) optionally, an alcoholic, aqueous, or hydroalcoholic topical carrier, wherein the composition is essentially free of compounds which enhance skin penetration of active agents.

17. The method of claim 16, wherein the topical skin care composition is selected from the group consisting of include bodywashes, soaps, skin or hair lotions, skin or hair sprays, sunscreens, shampoos and conditioners.

18. An article comprising i.) at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; ii.) optionally, at least one sensory compound; and optionally, an alcoholic, aqueous, or hydroalcoholic topical carrier, wherein the article is essentially free of compounds which enhance skin penetration of active agents.

19. The article of claim 18, wherein the article is selected from the group consisting of cleaning wipes, tissues, pads, disposable absorbent articles, adhesive articles, wraps and light shielding covers.

20. A method of reducing harsh or unpleasant sensations associated with the topical application of sensory compounds to an host's skin or mucosal membranes of the eye, otic cavity, nasal cavity, oral cavity, throat or vaginal area , comprising the step of:
a. providing a article comprising:
i. at least one soothing agent selected from the group consisting of saturated or unsaturated, straight or branched chain alkyls having a carbon backbone and having no more than carbons and wherein the alkyl has at least two hydroxyl groups such that a first hydroxyl group is attached to a first carbon of the carbon backbone and a second hydroxyl group is attached to a second carbon of the carbon backbone adjacent to the first carbon; and
ii. at least one sensory compound; and
iii. optionally, an alcoholic, aqueous, or hydroalcoholic topical carrier, wherein the article is essentially free of compounds which enhance skin penetration of active agents;
and
b. applying the article to the host's skin or mucosal membranes of the eye, oral cavity, nose, throat or vaginal area.
